(19) United States
(12) Patent Application Publication
(10) Pub. No.: US 2026/0041827 A1
(43) Pub. Date: Feb. 12, 2026

(54) SYSTEM AND METHOD FOR MONITORING PATIENT UNDERGOING HEMODIALYSIS TREATMENT

(71) Applicant: Taipei Medical University, Taipei City (TW)

(72) Inventor: Jian-Chiun Liou, Hsinchu City (TW)

(73) Assignee: Taipei Medical University, Taipei City (TW)

(21) Appl. No.: 18/978,914

(22) Filed: Dec. 12, 2024

(30) Foreign Application Priority Data
Aug. 12, 2024 (TW) 113130166

(51) Int. Cl. A61M 1/16

(52) U.S. Cl. CPC A61M 1/1601; A61M 2205/18; A61M 2230/06; A61M 2230/20; A61M 2230/65

(57) ABSTRACT

A system for monitoring a patient undergoing hemodialysis treatment includes a blood oxygen measurement device, a heart rate measurement device, an impedance measurement device and a monitoring device. The blood oxygen measurement device measures a blood oxygen level of the patient so as to generate a blood oxygen signal. The heart rate measurement device measures a heart rate of the patient so as to generate a heart rate signal. The impedance measurement device applies a first current signal and a second current signal to the patient, so as to obtain a first impedance signal and a second impedance signal respectively. The monitoring device receives the first impedance signal and the second impedance signal from the impedance measurement device, and emits a warning message when determining that a first impedance value of the first impedance signal is equal to a second impedance value of the second impedance signal.

### CROSS-REFERENCE TO RELATED APPLICATION

This application claims priority to Taiwanese Invention Patent Application No. 113130166, filed on Aug. 12, 2024, the entire disclosure of which is incorporated by reference herein.

### FIELD

The disclosure relates to a system and a method for monitoring a patient, and more particularly to a system and a method for monitoring a patient undergoing hemodialysis treatment.

### BACKGROUND

Hemodialysis is a treatment that drains blood out of a patient's body, removes metabolic wastes in the blood through a hemodialysis machine, and then returns the purified blood back into the patient's body. Hemodialysis patients usually have to go to the hospital 2 to 3 times a week to receive hemodialysis treatment. During the hemodialysis treatment, patients often experience muscle spasms, such as lower limb spasms or abdominal muscle spasms. Excessive dehydration is one of the main reasons for muscle spasms during hemodialysis treatment, which causes rapid changes in the body's osmotic pressure, and thus causes muscle spasms and contractions.

Current study shows that, before muscle spasms occur, physiological changes such as an increase in heart rate and/or a decrease in blood oxygen level can often be observed in patients. Therefore, some hospitals practice a method for determining whether to pause or stop the hemodialysis treatment by periodically measuring the heart rate and the blood oxygen level of the patient during the hemodialysis treatment, in the hope of reducing the occurrence of muscle spasms. However, such method is not ideal because not every patient would have an obvious increase in heart rate or an obvious decrease in blood oxygen level before muscle spasms occur.

### SUMMARY

Therefore, an object of the disclosure is to provide a system and a method for monitoring a patient undergoing hemodialysis treatment that can alleviate at least one of the drawbacks of the prior art.

According to an aspect of the disclosure, a system for monitoring a patient undergoing hemodialysis treatment includes a blood oxygen measurement device, a heart rate measurement device, an impedance measurement device and a monitoring device. The blood oxygen measurement device is configured to measure a blood oxygen level of the patient, and to generate a blood oxygen signal based on the blood oxygen level thus measured. The heart rate measurement device is configured to measure a heart rate of the patient, and to generate a heart rate signal based on the heart rate thus measured. The impedance measurement device includes a first impedance module and a second impedance module that are adapted to be placed on body parts of the patient. The impedance measurement device further includes a first controller that is configured to control the first impedance module to apply a first current signal to the patient, to perform impedance measurement through the first impedance module so as to obtain a first impedance signal, to control the second impedance module to apply a second current signal to the patient, and to perform impedance measurement through the second impedance module so as to obtain a second impedance signal. A frequency of the first current signal ranges from 5 kilohertz (kHz) to 10 kHz, and a frequency of the second current signal ranges from 200 kHz to 300 kHz. The monitoring device is electrically connected to the blood oxygen measurement device, the heart rate measurement device and the impedance measurement device, and includes a warning module and a second controller. The monitoring device receives the first impedance signal and the second impedance signal from the impedance measurement device, and the second controller is configured to, in response to determining that a first impedance value of the first impedance signal is equal to a second impedance value of the second impedance signal, control the warning module to emit a warning message.

According to another aspect of the disclosure, a method for monitoring a patient undergoing hemodialysis treatment is to be implemented by a system. The system includes an impedance measurement device, and a monitoring device electrically connected to the impedance measurement device. The method includes: the impedance measurement device measuring impedance of a limb of the patient so as to obtain a first impedance signal; the impedance measurement device measuring impedance of the chest of the patient so as to obtain a second impedance signal; and the monitoring device emitting a warning message in response to determining that a first impedance value of the first impedance signal is equal to a second impedance value of the second impedance signal.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 is a schematic view illustrating a system for monitoring a patient undergoing hemodialysis treatment according to an embodiment of the disclosure.

FIG. 2 is a block diagram illustrating the system according to an embodiment of the disclosure.

FIG. 3 includes plots illustrating changes in a blood oxygen value, a heart rate value, a first impedance value and a second impedance value over time.

FIG. 4 is a flow chart illustrating a method for monitoring a patient undergoing hemodialysis treatment according to an embodiment of the disclosure.

FIG. 5 is a flow chart illustrating sub-steps for obtaining a first impedance signal.

FIG. 6 is a flow chart illustrating sub-steps for obtaining a second impedance signal.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

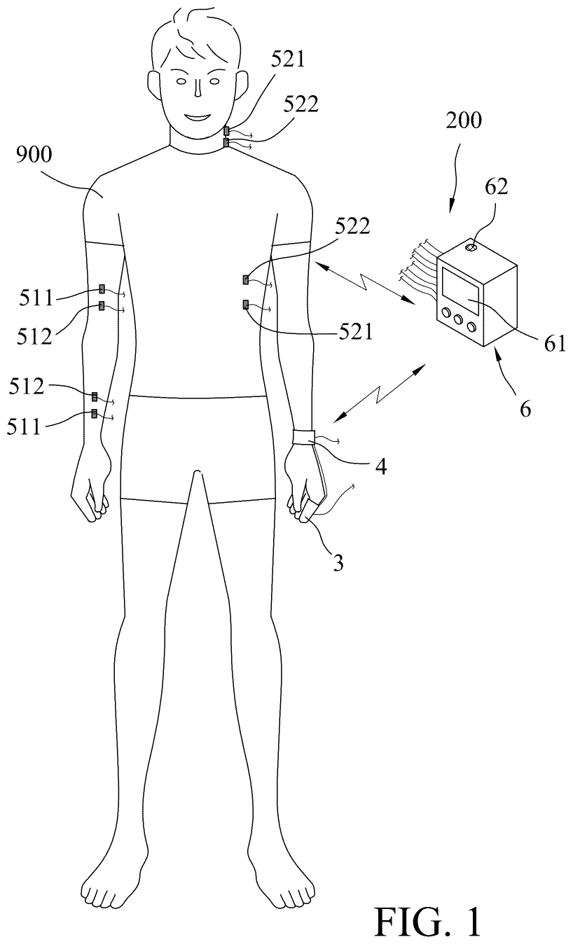

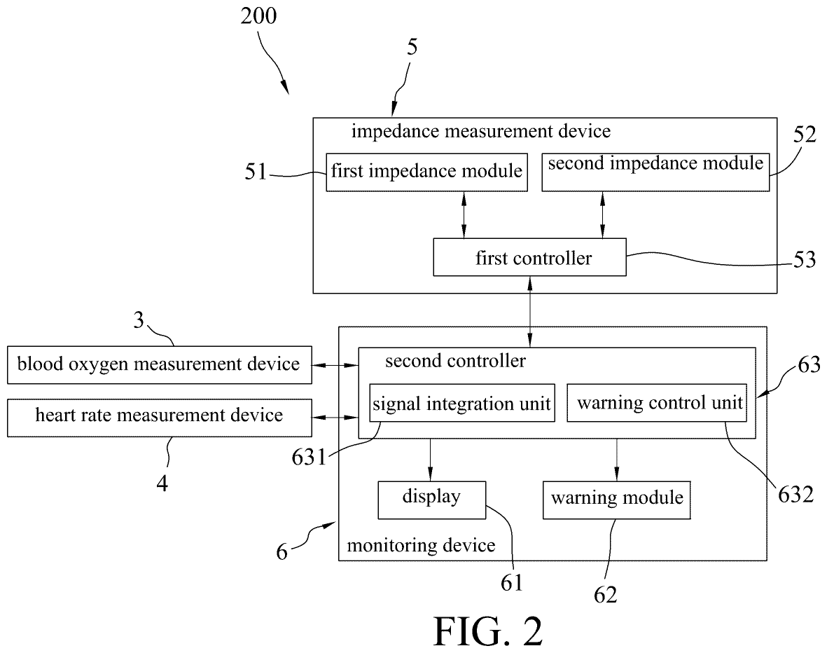

Referring to FIGS. 1 and 2, according to an embodiment of the disclosure, a system 200 for monitoring a patient 900 undergoing hemodialysis treatment is configured to monitor physiological data of the patient 900 during the hemodialysis treatment. The physiological data may be used as a reference for a medical staff to evaluate a body osmotic pressure of the patient 900, which may be useful for the medical staff to evaluate the potential for occurrence of muscle spasms more accurately.

The system 200 includes a blood oxygen measurement device 3, a heart rate measurement device 4, an impedance measurement device 5 and a monitoring device 6. The monitoring device 6 is electrically connected to the blood oxygen measurement device 3, the heart rate measurement device 4 and the impedance measurement device 5.

The blood oxygen measurement device 3 is adapted to be placed on a finger of the patient 900, and is configured to measure a blood oxygen level of the patient 900 over time, and to generate a blood oxygen signal based on the blood oxygen level thus measured. The heart rate measurement device 4 is adapted to be wrapped around a wrist of the patient 900, and is configured to measure a heart rate of the patient 900 over time, and to generate a heart rate signal based on the heart rate thus measured. It should be noted that the blood oxygen signal has a blood oxygen value that varies over time, and the heart rate signal has a heart rate value that varies over time. In this embodiment, the blood oxygen measurement device 3 and the heart rate measurement device 4 are two separate devices. In some embodiments, the blood oxygen measurement device 3 and the heart rate measurement device 4 may be integrated into a single wearable device, such as a watch, but the disclosure is not limited to such. Since the blood oxygen measurement device 3 and the heart rate measurement device 4 are both existing technologies and may be implemented in various ways, they will not be described in further detail for the sake of brevity.

The impedance measurement device 5 includes a first impedance module 51 that is configured to measure impedance of a limb of the patient 900 (hereinafter referred to as “limb impedance”), a second impedance module 52 that is configured to measure impedance of the chest of the patient 900 (hereinafter referred to as “chest impedance”), and a first controller 53 that is electrically connected to the first impedance module 51 and the second impedance module 52. In this embodiment, the first impedance module 51 is adapted to be placed on an arm of the patient 900 so as to measure the limb impedance, and the second impedance module 52 is adapted to be placed on the neck and the chest of the patient 900 so as to measure the chest impedance.

The first impedance module 51 includes two first transmitting electrodes 511 and two first measuring electrodes 512. The first transmitting electrodes 511 are adapted to be placed on and spaced apart from each other along the arm of the patient 900 (e.g., placed respectively on an upper arm and a lower arm of the patient 900). The first measuring electrodes 512 are adapted to be placed on and spaced apart from each other along the arm of the patient 900 (e.g., placed respectively on the upper arm and the lower arm of the patient 900), and are disposed between the first transmitting electrodes 511.

The second impedance module 52 includes two second transmitting electrodes 521 and two second measuring electrodes 522. The second transmitting electrodes 521 are adapted to be placed respectively on the neck and the chest of the patient 900, and the second measuring electrodes 522 are adapted to be placed respectively on the neck and the chest of the patient 900 and are disposed between the second transmitting electrodes 521.

The first controller 53 is configured to control the first transmitting electrodes 511 to apply a first current signal to the limb of the patient 900, and is further configured to perform impedance measurement through the first measuring electrodes 512 so as to obtain a first impedance signal, thus measuring the limb impedance of the patient 900. For example, the first controller 53 may measure a resulting voltage across the first measuring electrodes 512, and calculate the limb impedance based on the first current signal and the resulting voltage thus measured. The first controller 53 is further configured to control the second transmitting electrodes 521 to apply a second current signal to the patient 900, and is further configured to perform impedance measurement through the second measuring electrodes 522 so as to obtain a second impedance signal, thus measuring the chest impedance of the patient 900. For example, the first controller 53 may measure a resulting voltage across the second measuring electrodes 522, and calculate the chest impedance based on the second current signal and the resulting voltage thus measured. It should be noted that, the first impedance signal has a first impedance value that varies over time, and the second impedance signal has a second impedance value that varies over time. In this embodiment, the first controller 53 is integrated into the monitoring device 6, but the disclosure is not limited to such.

A frequency of the first current signal ranges from 5 kilohertz (kHz) to 10 kHz, and a frequency of the second current signal ranges from 200 kHz to 300 kHz. Both a current value of the first current signal and a current value of the second current signal range from 1 milliampere (mA) to 3 mA. In this embodiment, the frequency of the first current signal is 8 kHz, and the frequency of the second current signal is 250 kHz.

During the hemodialysis treatment, by placing the first transmitting electrodes 511 and the first measuring electrodes 512 on the arm of the patient 900, and applying the first current signal with the frequency ranging from 5 kHz to 10 kHz, the limb impedance of the patient 900 over time may be measured. In other words, changes in an osmotic pressure of the limb of the patient 900 over time that are caused by dehydration may be measured.

During the hemodialysis treatment, by placing the second transmitting electrodes 521 and the second measuring electrodes 522 on the neck and the chest of the patient 900, and applying the second current signal with the frequency ranging from 200 kHz to 300 kHz, the chest impedance in the vicinity of the heart of the patient 900 over time may be measured. In other words, changes in an osmotic pressure in the vicinity of the heart of the patient 900 over time that are caused by dehydration may be measured.

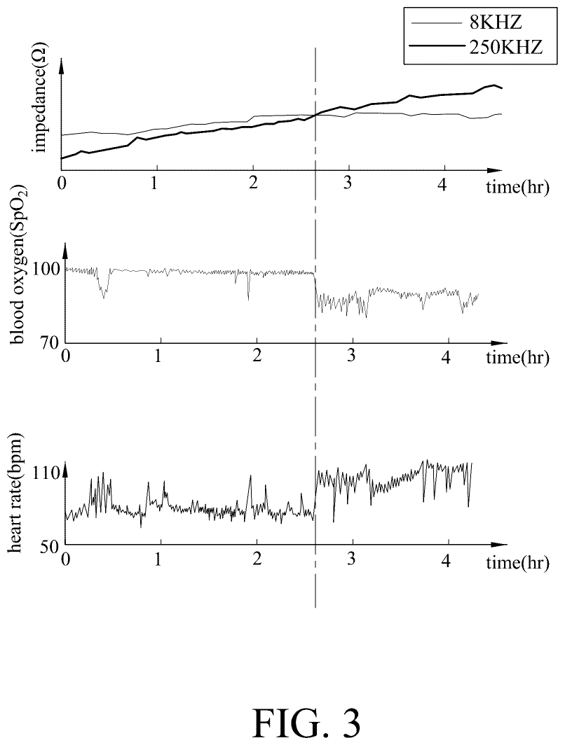

Referring further to FIG. 3, the monitoring device 6 includes a display 61, a warning module 62, and a second controller 63 (see FIG. 2) that is electrically connected to the display 61 and the warning module 62. The second controller 63 is further electrically connected to the blood oxygen measurement device 3, the heart rate measurement device 4 and the first controller 53, and is configured to receive the blood oxygen signal from the blood oxygen measurement device 3, to receive the heart rate signal from the heart rate measurement device 4, and to receive the first impedance signal and the second impedance signal from the first controller 53.

The second controller 63 includes a signal integration unit 631 and a warning control unit 632. In this embodiment, each of the signal integration unit 631 and the warning control unit 632 may include one or more of, but is not limited to, a single core processor, a multi-core processor, a dual-core mobile processor, a microprocessor, a microcontroller, a digital signal processor (DSP), a field-programmable gate array (FPGA), an application specific integrated circuit (ASIC), a radio-frequency integrated circuit (RFIC), a system on a chip (SoC), etc.

The signal integration unit 631 is configured to obtain the blood oxygen value of the blood oxygen signal, the heart rate value of the heart rate signal, the first impedance value of the first impedance signal, and the second impedance value of the second impedance signal, over time. The signal integration unit 631 is further configured to control the display 61 to simultaneously display the following thereon: 1) a blood oxygen plot illustrating the blood oxygen value of the blood oxygen signal over time; 2) a heart rate plot illustrating the heart rate value of the heart rate signal over time; and 3) an impedance plot illustrating the first impedance value of the first impedance signal over time and the second impedance value of the second impedance signal over time.

Researchers have discovered through experiments that before a patient 900 experiences muscle spasms during the hemodialysis treatment, in addition to an increase in heart rate and a decrease in blood oxygen level, a gradual increase in impedance of the body (hereinafter referred to as “body impedance”) due to dehydration would also be observed in the patient 900. Since the degrees of dehydration near the limbs of the patient 900 and near the heart of the patient 900 are different, the first impedance value and the second impedance value would gradually increase in different manners. Additionally, before the patient 900 experiences muscle spasms, the first impedance value and the second impedance value would be equal to each other at one time point (i.e., the impedance plot shows an intersection at one time point). Therefore, warnings for potential muscle spasms may be done by analyzing the first impedance value and the second impedance value.

The warning control unit 632 is configured to, in response to determining that the first impedance value is equal to the second impedance value at a current time point (i.e., when a line representing the first impedance value intersects a line representing the second impedance value on the impedance plot), control the warning module 62 to emit a warning message. In one example, the warning module 62 includes a display, a lighting device, an audio device, or a combination thereof, and emits the warning message in the form of an image, light, sound or any combination thereof.

As such, when the warning module 62 emits the warning message, it means that the limb impedance and the chest impedance of the patient 900 have increased to, and the body osmotic pressure of the patient 900 has decreased to, a point where muscle spasms would likely occur during subsequent hemodialysis treatment. The patient 900 or the medical staff may then evaluate whether to pause or stop the hemodialysis treatment based on the warning message and the plots on the display 61, so as to reduce the occurrence of muscle spasms.

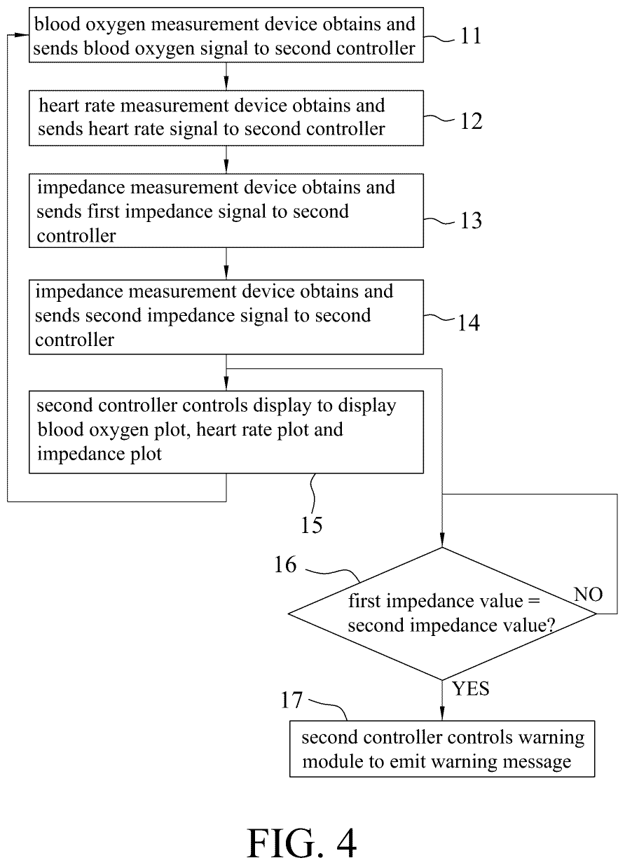

Referring further to FIG. 4, a method for monitoring the patient 900 undergoing hemodialysis treatment is implemented by the system 200, and a flow of the method includes steps 11 to 17.

In step 11, the blood oxygen measurement device 3 measures the blood oxygen level of the patient 900 over time so as to obtain the blood oxygen signal, and sends the blood oxygen signal to the second controller 63.

In step 12, the heart rate measurement device 4 measures the heart rate of the patient 900 over time so as to obtain the heart rate signal, and sends the heart rate signal to the second controller 63.

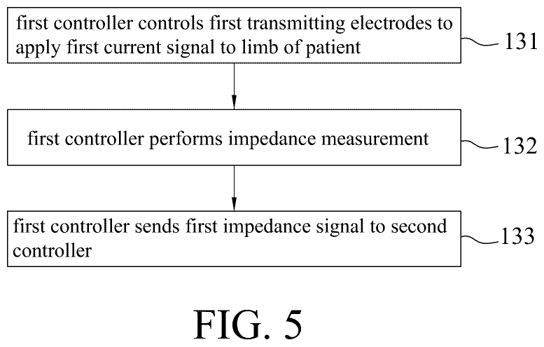

In step 13, the impedance measurement device 5 measures the limb impedance of the patient 900 over time so as to obtain the first impedance signal, and sends the first impedance signal to the second controller 63. To describe in further detail, step 13 includes sub-steps 131 to 133 (see FIG. 5).

In sub-step 131, the first controller 53 controls the first transmitting electrodes 511 to apply the first current signal to the limb of the patient 900, where the frequency of the first current signal ranges from 5 kHz to 10 kHz, and the current value of the first current signal ranges from 1 mA to 3 mA.

In sub-step 132, the first controller 53 performs impedance measurement through the first measuring electrodes 512 so as to obtain the first impedance signal.

In sub-step 133, the first controller 53 sends the first impedance signal to the second controller 63.

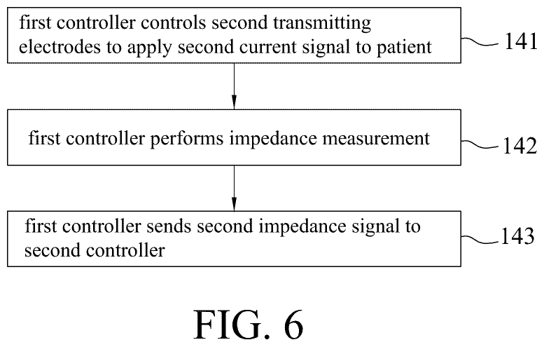

In step 14, the impedance measurement device 5 measures the chest impedance of the patient 900 over time so as to obtain the second impedance signal, and sends the second impedance signal to the second controller 63. To describe in further detail, step 14 includes sub-steps 141 to 143 (see FIG. 6).

In sub-step 141, the first controller 53 controls the second transmitting electrodes 521 to apply the second current signal to the patient 900, where the frequency of the second current signal ranges from 200 kHz to 300 kHz, and the current value of the second current signal ranges from 1 mA to 3 mA.

In sub-step 142, the first controller 53 performs impedance measurement through the second measuring electrodes 522 so as to obtain the second impedance signal.

In sub-step 143, the first controller 53 sends the second impedance signal to the second controller 63.

It should be noted that steps 11 to 14 may be performed simultaneously or may be switched in order as long as the signals are obtained and sent to the second controller 63 before step 15 is performed.

In step 15, the second controller 63 controls the display 61 to simultaneously display thereon the blood oxygen plot, the heart rate plot and the impedance plot, based on the blood oxygen signal, the heart rate signal, the first impedance signal and the second impedance signal received in steps 11 to 14, and the flow goes back to step 11.

In step 16, the second controller 63 determines whether the first impedance value is equal to the second impedance value at a current time point, and when a result of the determination is affirmative, the flow proceeds to step 17. When the result of the determination is negative, the second controller 63 repeats step 16 for determining whether the first impedance value and the second impedance value are equal to each other at a next time point (e.g., 1 second later).

In step 17, the second controller 63 controls the warning module 62 to emit the warning message.

It should be noted that steps 11 to 17 and the flow chart shown in FIG. 4 merely constitute one example of the disclosure, and steps 11 to 17 may be combined, divided, or switched in order as long as the method under such adjustment achieves substantially the same function in substantially the same way as provided in the embodiment.

In summary, according to the disclosure, during the hemodialysis treatment, the impedance measurement device 5 simultaneously measures the first impedance value (i.e., the limb impedance) and the second impedance value (i.e., the chest impedance) of the patient 900, and the monitoring device 6 displays the impedance plot on the display 61 and sends out the warning message through the warning module 62 when determining that the first impedance value is equal to the second impedance value at a current time point. As such, the body impedance (which is related to the body osmotic pressure) of the patient 900 may be monitored in real time, and the plots displayed on the display 61 may be referred to by the medical staff to evaluate whether to pause or stop the hemodialysis treatment. Compared to predicting muscle spasms through monitoring the blood oxygen level and the heart rate only, changes in which may sometimes be unobvious, the method and the system 200 provided in the disclosure allow the medical staff to evaluate the potential for occurrence of muscle spasms on the patient 900 more accurately.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A system for monitoring a patient undergoing hemodialysis treatment, comprising:
a blood oxygen measurement device configured to measure a blood oxygen level of the patient, and to generate a blood oxygen signal based on the blood oxygen level thus measured;
a heart rate measurement device configured to measure a heart rate of the patient, and to generate a heart rate signal based on the heart rate thus measured;
an impedance measurement device including a first impedance module and a second impedance module that are adapted to be placed on body parts of the patient, and a first controller that is configured to control said first impedance module to apply a first current signal to the patient, to perform impedance measurement through said first impedance module so as to obtain a first impedance signal, to control said second impedance module to apply a second current signal to the patient, and to perform impedance measurement through said second impedance module so as to obtain a second impedance signal, where a frequency of the first current signal ranges from 5 kilohertz (kHz) to 10 kHz, and a frequency of the second current signal ranges from 200 kHz to 300 kHz; and
a monitoring device electrically connected to said blood oxygen measurement device, said heart rate measurement device and said impedance measurement device, and including a warning module and a second controller, said monitoring device receiving the first impedance signal and the second impedance signal from said impedance measurement device, said second controller being configured to, in response to determining that a first impedance value of the first impedance signal is equal to a second impedance value of the second impedance signal, control said warning module to emit a warning message.

2. The system as claimed in claim 1, wherein:
said monitoring device further includes a display;
said monitoring device receives the blood oxygen signal from said blood oxygen measurement device, and receives the heart rate signal from said heart rate measurement device;
said second controller includes a signal integration unit that is configured to control said display to display thereon a blood oxygen value of the blood oxygen signal over time, a heart rate value of the heart rate signal over time, the first impedance value of the first impedance signal over time and the second impedance value of the second impedance signal over time; and
said second controller further includes a warning control unit that is configured to, in response to determining that the first impedance value is equal to the second impedance value at a current time point, control said warning module to emit the warning message.

3. The system as claimed in claim 1, wherein the frequency of the first current signal is 8 kHz, and the frequency of the second current signal is 250 kHz.

4. The system as claimed in claim 3, wherein both a current value of the first current signal and a current value of the second current signal range from 1 milliampere (mA) to 3 mA.

5. The system as claimed in claim 1, wherein both a current value of the first current signal and a current value of the second current signal range from 1 mA to 3 mA.

6. The system as claimed in claim 1, wherein:
said first impedance module includes two first transmitting electrodes that are adapted to be placed on and spaced apart from each other along an arm of the patient, and two first measuring electrodes that are adapted to be placed on and spaced apart from each other along the arm of the patient and that are disposed between said first transmitting electrodes; and
said first controller is configured to control said first transmitting electrodes to apply the first current signal, and to perform the impedance measurement through said first measuring electrodes so as to obtain the first impedance signal.

7. The system as claimed in claim 6, wherein:
said second impedance module includes two second transmitting electrodes that are adapted to be placed respectively on a neck and a chest of the patient, and two second measuring electrodes that are adapted to be placed respectively on the neck and the chest of the patient and that are disposed between said second transmitting electrodes; and
said first controller is configured to control said second transmitting electrodes to apply the second current signal, and to perform the impedance measurement through said second measuring electrodes so as to obtain the second impedance signal.

8. The system as claimed in claim 1, wherein:
said second impedance module includes two second transmitting electrodes that are adapted to be placed respectively on a neck and a chest of the patient, and two second measuring electrodes that are adapted to be placed respectively on the neck and the chest of the patient and that are disposed between said second transmitting electrodes; and
said first controller is configured to control said second transmitting electrodes to apply the second current signal, and to perform the impedance measurement through said second measuring electrodes so as to obtain the second impedance signal.

9. A method for monitoring a patient undergoing hemodialysis treatment, the method to be implemented by a system, the system including an impedance measurement device, and a monitoring device electrically connected to the impedance measurement device, the method comprising:
the impedance measurement device measuring impedance of a limb of the patient so as to obtain a first impedance signal;
the impedance measurement device measuring impedance of a chest of the patient so as to obtain a second impedance signal; and
the monitoring device emitting a warning message in response to determining that a first impedance value of the first impedance signal is equal to a second impedance value of the second impedance signal.

10. The method as claimed in claim 9, wherein:
measuring impedance of a limb of the patient includes applying a first current signal to the limb of the patient, where a frequency of the first current signal ranges from 5 kHz to 10 kHz; and
measuring impedance of a chest of the patient includes applying a second current signal to the patient, where a frequency of the second current signal ranges from 200 kHz to 300 KHz.

11. The method as claimed in claim 10, wherein both a current value of the first current signal and a current value of the second current signal range from 1 mA to 3 mA.

12. The method as claimed in claim 11, wherein the frequency of the first current signal is 8 kHz, and the frequency of the second current signal is 250 k Hz.

13. The method as claimed in claim 10, wherein the frequency of the first current signal is 8 kHz, and the frequency of the second current signal is 250 KHz.
